(19) United States
(12) Patent Application Publication
(10) Pub. No.: US 2026/0155243 A1
(43) Pub. Date: Jun. 4, 2026

(54) SYSTEMS AND METHODS FOR AGENTIC CLINICAL WORKFLOW GOVERNANCE WITH DETERMINISTIC HUMAN ESCALATION BOUNDARIES

(71) Applicant: George William Bickerstaff, III, Greenwich, CT (US)

(72) Inventor: George William Bickerstaff, III, Greenwich, CT (US)

(21) Appl. No.: 19/456,442

(22) Filed: Jan. 22, 2026

(51) Int. Cl. G16H 40/20; G06Q 10/0633

(52) U.S. Cl. CPC G16H 40/20; G06Q 10/0633

(57) ABSTRACT

Systems and methods for agentic clinical workflow governance with deterministic human escalation boundaries are disclosed. A hardware-isolated governance engine enforces consensus, risk, and regulatory constraints across multi-agent clinical workflows, deterministically freezing execution and escalating control to verified human oversight when required.

### TECHNICAL FIELD

The present invention relates to regulated clinical workflows involving artificial intelligence agents, robotic systems, and computer-assisted decision support. More particularly, the invention relates to deterministic governance of multi-agent clinical workflows with enforced human escalation boundaries in regulated medical environments.

### BACKGROUND

Clinical workflows increasingly rely on distributed artificial intelligence agents to assist with diagnosis, treatment planning, monitoring, documentation, and procedural execution. These agents may operate autonomously or semi-autonomously across different stages of patient care.

Existing clinical workflow orchestration systems lack deterministic controls ensuring that autonomous agents act only within approved clinical, regulatory, and institutional boundaries. As agentic systems become more complex, emergent behavior and inter-agent conflicts create safety, compliance, and liability risks.

Current escalation mechanisms are often advisory, discretionary, or post hoc, failing to guarantee that ambiguous, conflicting, or high-risk agent decisions are routed to qualified human oversight before execution. This gap exposes institutions to regulatory violations and patient harm.

Accordingly, there exists a need for a hardware-isolated governance system that deterministically enforces agentic workflow constraints, detects non-concordance, freezes execution, and escalates control to verified human oversight at predefined boundaries.

### SUMMARY

The disclosed invention provides systems and methods for agentic clinical workflow governance with deterministic human escalation boundaries. A hardware-isolated governance engine orchestrates multi-agent clinical workflows while enforcing predefined consensus, scope, and compliance constraints.

The governance engine continuously evaluates agent outputs for concordance, risk thresholds, and regulatory compliance. Upon detection of non-concordance, ambiguity, or violation, workflow execution is deterministically frozen and routed to verified human oversight.

All agent actions, governance decisions, escalations, and resolutions are recorded in immutable provenance records, enabling regulatory compliance, liability attribution, and institutional audit.

### DEFINITIONS

Agentic Workflow means a coordinated sequence of actions performed by multiple artificial intelligence agents within a clinical process.

Governance Engine means a hardware-isolated computational component enforcing workflow constraints and escalation rules.

Non-Concordance means disagreement, inconsistency, or conflict among agent outputs or between agents and predefined constraints.

Escalation Boundary means a deterministic checkpoint requiring human oversight prior to continued execution.

Human Oversight means intervention by a verified clinician or authorized personnel.

Workflow Freeze means deterministic suspension of further agent actions.

Consensus Threshold means a predefined level of agreement required among agents.

Immutable Provenance Record means a tamper-resistant record of workflow events.

Regulatory Constraint means a rule derived from law, regulation, or institutional policy.

Execution Context means the clinical, temporal, and regulatory state in which agents operate.

### BRIEF DESCRIPTION OF THE DRAWINGS

FIG. 1—AGENTIC WORKFLOW ARCHITECTURE illustrates governance of multi-agent clinical workflows.

FIG. 2—CONSENSUS AND CONCORDANCE EVALUATION illustrates agent agreement and conflict detection.

FIG. 3—DETERMINISTIC ESCALATION BOUNDARY illustrates workflow freeze and human routing.

FIG. 4—HUMAN OVERSIGHT RESOLUTION illustrates authorized human decision-making.

FIG. 5—PROVENANCE AND COMPLIANCE UTILIZATION illustrates downstream audit and regulatory use.

### DETAILED DESCRIPTION OF THE DRAWINGS

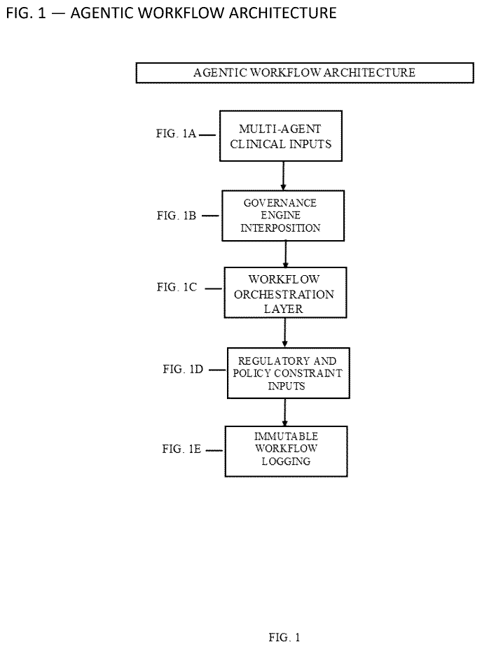

### FIG. 1—Agentic Workflow Architecture

FIG. 1A—MULTI-AGENT CLINICAL INPUTS illustrates multiple AI agents generating diagnostic, planning, or monitoring outputs. Each agent operates within a defined scope. Outputs are timestamped and contextualized.

FIG. 1B—GOVERNANCE ENGINE INTERPOSITION illustrates a hardware-isolated governance engine receiving agent outputs. The engine operates independently of agent logic. Bypass is prevented.

FIG. 1C—WORKFLOW ORCHESTRATION LAYER illustrates controlled sequencing of agent actions. Execution depends on governance approval. Unauthorized transitions are blocked.

FIG. 1D—REGULATORY AND POLICY CONSTRAINT INPUTS illustrates ingestion of regulatory, institutional, and procedural constraints. Constraints are machine-enforceable. Updates propagate deterministically.

FIG. 1E—IMMUTABLE WORKFLOW LOGGING illustrates recording of workflow events into an immutable ledger. Integrity is preserved. Records are independently verifiable.

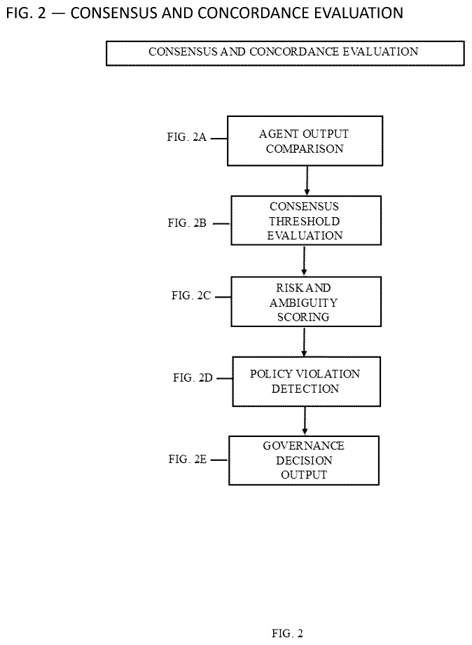

### FIG. 2—Consensus and Concordance Evaluation

FIG. 2A—AGENT OUTPUT COMPARISON illustrates comparison of outputs across agents. Agreement levels are computed. Discrepancies are identified.

FIG. 2B—CONSENSUS THRESHOLD EVALUATION illustrates application of predefined consensus thresholds. Threshold failure triggers escalation conditions. Execution does not proceed.

FIG. 2C—RISK AND AMBIGUITY SCORING illustrates risk scoring applied to agent outputs. Ambiguity metrics are computed. High-risk states are flagged.

FIG. 2D—POLICY VIOLATION DETECTION illustrates detection of regulatory or institutional violations. Violations override consensus success. Deterministic block occurs.

FIG. 2E—GOVERNANCE DECISION OUTPUT illustrates generation of approve, block, or escalate decisions. Decisions are final within the execution cycle. Provenance is recorded.

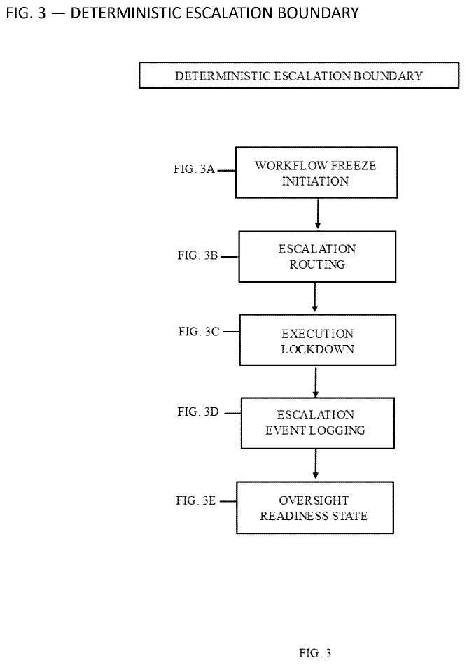

### FIG. 3—Deterministic Escalation Boundary

FIG. 3A—WORKFLOW FREEZE INITIATION illustrates immediate suspension of agent actions upon escalation trigger. State is preserved. Patient safety is maintained.

FIG. 3B—ESCALATION ROUTING illustrates routing of workflow context to verified human oversight. Full provenance accompanies escalation. Context loss is prevented.

FIG. 3C—EXECUTION LOCKDOWN illustrates enforcement of non-bypassable execution lock. No agent may proceed. Lock integrity is maintained.

FIG. 3D—ESCALATION EVENT LOGGING illustrates immutable recording of escalation events. Time, reason, and context are captured. Auditability is preserved.

FIG. 3E—OVERSIGHT READINESS STATE illustrates system readiness pending human resolution. Systems remain stable. Unauthorized actions are prevented.

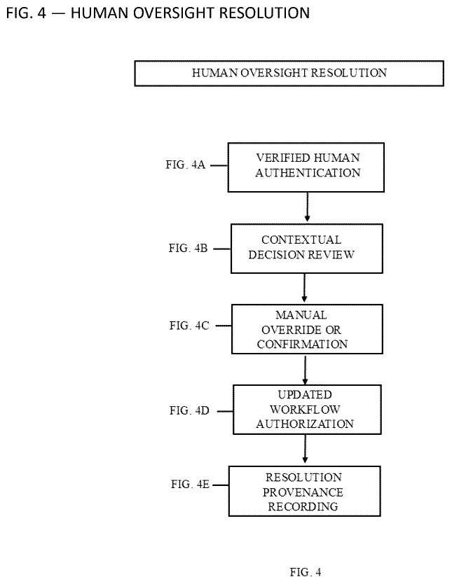

### FIG. 4—Human Oversight Resolution

FIG. 4A—VERIFIED HUMAN AUTHENTICATION illustrates authentication of oversight personnel. Credentials and scope are verified. Unauthorized access is denied.

FIG. 4B—CONTEXTUAL DECISION REVIEW illustrates presentation of full workflow context to human oversight. Agent outputs and constraints are visible. Decisions are informed.

FIG. 4C—MANUAL OVERRIDE OR CONFIRMATION illustrates human confirmation, modification, or override of agent recommendations. Decisions are explicit. Scope is enforced.

FIG. 4D—UPDATED WORKFLOW AUTHORIZATION illustrates issuance of updated authorization allowing workflow continuation. Prior restrictions are reconciled. Execution resumes deterministically.

FIG. 4E—RESOLUTION PROVENANCE RECORDING illustrates recording of oversight decisions into immutable records. Accountability is preserved. Traceability is ensured.

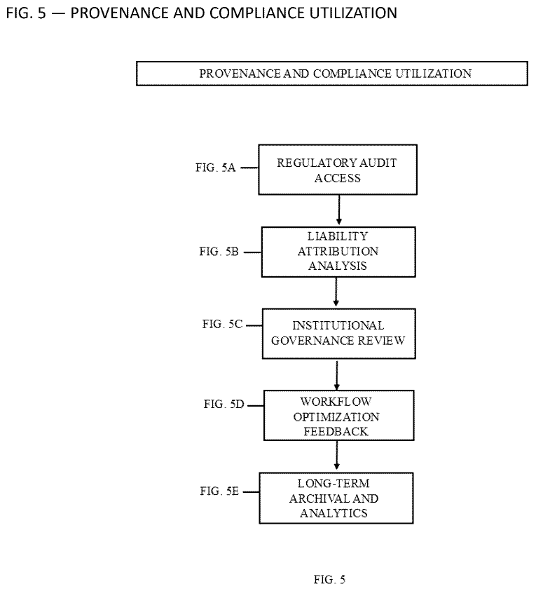

### FIG. 5—Provenance and Compliance Utilization

FIG. 5A—REGULATORY AUDIT ACCESS illustrates regulator review of governance and escalation records. Compliance is independently verifiable. Records are immutable.

FIG. 5B—LIABILITY ATTRIBUTION ANALYSIS illustrates attribution of responsibility across agents and humans. Causality is clarified. Disputes are reduced.

FIG. 5C—INSTITUTIONAL GOVERNANCE REVIEW illustrates hospital oversight of agentic workflows. Policy enforcement is verified. Quality improvement is supported.

FIG. 5D—WORKFLOW OPTIMIZATION FEEDBACK illustrates use of provenance data to refine workflow design. Safety improves over time. Errors decrease.

FIG. 5E—LONT-TEM ARCHIVAL AND ANALYTICS illustrates archival and longitudinal analysis of governance data. Integrity persists. Research is enabled.

### EXAMPLES

### Example 1—Diagnostic Agent Disagreement

Multiple diagnostic agents produce conflicting treatment recommendations. Consensus threshold fails. Workflow freezes and escalates to a physician, who selects the appropriate path.

### Example 2—High-Risk Treatment Plan

An AI planning agent proposes a high-risk intervention. Risk threshold triggers escalation. Human oversight modifies the plan before execution.

1. A system for governing agentic clinical workflows, comprising:
a hardware-isolated governance engine configured to evaluate outputs from multiple artificial intelligence agents;
a consensus and risk evaluation module enforcing predefined thresholds; and
a deterministic escalation boundary freezing workflow execution and routing control to verified human oversight upon non-concordance.

2. A computer-implemented method comprising:
receiving outputs from multiple clinical AI agents;
evaluating consensus, risk, and regulatory compliance;
freezing workflow execution upon violation or ambiguity; and
escalating control to verified human oversight.

3. A non-transitory computer-readable medium storing instructions that, when executed by a hardware-isolated governance engine, cause the system to perform the method of claim 2.

4. The system of claim 1, wherein the governance engine operates within a trusted execution environment.

5. The system of claim 1, wherein escalation events are immutably logged.

6. The system of claim 1, wherein workflow freeze is non-bypassable.

7. The method of claim 2, wherein consensus thresholds are dynamically configurable.

8. The method of claim 2, wherein escalation includes contextual data routing.

9. The system of claim 1, wherein regulatory constraints override agent consensus.

10. The non-transitory computer-readable medium of claim 3, further storing instructions for compliance audit support.
